(19) United States
(12) Patent Application Publication
(10) Pub. No.: US 2023/0178188 A1
(43) Pub. Date: Jun. 8, 2023

(54) METHOD, DEVICE, AND MEDIUM FOR GENERATING THREE-DIMENSION MOLECULE

(71) Applicant: Beijing Youzhuju Network Technology Co., Ltd., Beijing (CN)

(72) Inventors: Yi ZHOU, Beijing (CN); Siyu LONG, Beijing (CN)

(21) Appl. No.: 18/162,433

(22) Filed: Jan. 31, 2023

(51) Int. Cl. G16C 20/50; G16B 30/10; G16C 20/64; G16C 20/30; G06T 15/08

(52) U.S. Cl. CPC G16C 20/50; G16B 30/10; G16C 20/64; G16C 20/30; G06T 15/08

(57) ABSTRACT

Method, device and medium are directed to providing a method for generating three-dimension molecules. The method comprises obtaining a molecular shape of a three-dimension molecule for a drug, wherein the molecular shape is represented by a three-dimension image and generating a plurality of fragments of the three-dimension molecule based on the molecular shape. The method further comprises generating the three-dimension molecule by connecting the plurality of fragments. The method may be used to design high quality drugs for specific protein pockets efficiently and speed up the process of drug development and reduce the cycle of drug development. Furthermore, since the method utilizes large-scale non-experimental data, the method may not rely on expensive experimental data and docking simulation which is time consuming. Additionally, the method utilizes the three-dimension interaction information between molecules and pockets to generate drug molecules, and thus the quality of generated drug molecules can be improved.

### BACKGROUND

Drug design (often referred to as rational drug design or simply rational design) is the creative process of finding new drugs based on knowledge of biological targets. The drug is most commonly a small organic molecule that activates or inhibits the function of a biological molecule, such as a protein, resulting in a therapeutic benefit to the patient. Drug design involves the design of molecules with complementary shapes and charges. The molecules combine and interact with biological targets. Drug design often but not necessarily relies on computer modeling techniques. This type of modeling is sometimes called computer-aided drug design.

With the development of computer technology and computational chemistry, molecular biology and medicinal chemistry, drug design has entered a rational stage, and drug molecular design is the main direction of new drug discovery. It is based on the research results of life sciences such as biochemistry, enzymology, molecular biology and genetics, and aims at the potential drug design targets revealed in these basic studies, including enzymes, receptors, ion channels and nucleic acids, and refers to design rational drug molecules based on the chemical structure characteristics of other analogous ligands or natural products. The structure of a molecule may be described at various levels of sophistication. For example, the molecular formula tells which elements are present and in what ratio. The relative overall size and three-dimension shape give additional detail. Functional groups in the molecule indicate electrostatic properties. A description of chirality is necessary to account for the spatial relationship of the atoms. A further piece of information at a still higher level of sophistication would be the heat of formation.

### SUMMARY

In accordance with examples of the present disclosure, a method for designing drugs is described. The method comprises obtaining a molecular shape of a three-dimension molecule for a drug, wherein the molecular shape is represented by a three-dimension image. The method further comprises generating a plurality of fragments of the three-dimension molecule based on the molecular shape. The method further comprises generating the three-dimension molecule by connecting the plurality of fragments.

In accordance with the examples of the present disclosure, an electronic device comprising a memory and a processor is described. In examples, the memory is used to store one or more computer instructions which, when executed by the processor, cause the processor to: obtain a molecular shape of a three-dimension molecule for a drug, wherein the molecular shape is represented by a three-dimension image; generate a plurality of fragments of the three-dimension molecule based on the molecular shape; and generate the three-dimension molecule by connecting the plurality of fragments.

In accordance with the example of the present disclosure, a non-transitory computer-readable medium including instructions stored thereon which, when executed by an apparatus, cause the apparatus to perform acts including: obtaining a molecular shape of a three-dimension molecule for a drug, wherein the molecular shape is represented by a three-dimension image; generating a plurality of fragments of the three-dimension molecule based on the molecular shape; and generating the three-dimension molecule by connecting the plurality of fragments.

Any of the one or more above aspects in combination with any other of the one or more aspects. Any of the one or more aspects as described herein.

This Summary is provided to introduce a selection of concepts in a simplified form, which is further described below in the Detailed Description. This Summary is not intended to identify key features or essential features of the claimed subject matter, nor is it intended to be used to limit the scope of the claimed subject matter. Additional aspects, features, and/or advantages of examples will be set forth in part in the following description and, in part, will be apparent from the description, or may be learned by practice of the disclosure.

### BRIEF DESCRIPTION OF THE DRAWINGS

To easily identify the discussion of any particular element or act, the most significant digit or digits in a reference number refer to the figure number in which that element is first introduced.

FIG. 1 illustrates a schematic process hat splits a drug design process into sketching and generating stages;

FIG. 2A-C illustrate a training process for SHAPE2MOL model, example drug design process with SHAPE2MOL model based on ligand and example drug design process with SHAPE2MOL model based on pocket in accordance with embodiments of the present disclosure;

FIG. 3 illustrates an example process for sampling molecular shapes from pocket in accordance with embodiments of the present disclosure;

FIG. 4 illustrates architecture of SHAPE2MOL model with shape encoder and 3D molecule decoder;

FIG. 5 illustrates an example process for converting a molecule into a sequence in accordance with embodiments of the present disclosure;

FIG. 6 illustrates a flowchart of a method for designing drug that splits a drug design process into sketching and generating stages;

FIG. 7 is a block diagram illustrating physical components (for example hardware) of an electronic device with which aspects of the disclosure may be practiced.

### DETAILED DESCRIPTION

In the following detailed description, references are made to the accompanying drawings that form a part hereof, and in which are shown by way of illustrations specific aspects or examples. These aspects may be combined, other aspects may be utilized, and structural changes may be made without departing from the present disclosure. Aspects may be practiced as methods, systems or devices. Accordingly, aspects may take the form of a hardware implementation, an entirely software implementation, or an implementation combining software and hardware aspects. The following detailed description is therefore not to be taken in a limiting sense, and the scope of the present disclosure is defined by the appended claims and their equivalents.

Traditional drug design approaches usually employ virtual screening and molecular dynamics to traverse in a large scaled drug library, which is time consuming and could not produce novel drug candidates. Some drug design approaches utilize machine learning model rely on expensive experimental data while ignoring the 3D interaction information between drug and pocket. Other drug design approaches utilizing docking simulation is time consuming as well and the accuracy is not good enough.

Drug design is a crucial step in the drug discovery field, which is the process of finding new drugs based on a biological target. However, seeking appropriate drugs for a particular target is quite challenging due to the enormous space of drug candidates (almost 10³³). Traditional drug design approaches usually employ virtual screening and molecular dynamics to traverse in a large scaled drug library, which is time-consuming and could not produce novel drug candidates. Recently, a line of work proposes to realize drug design by generating drug molecules from scratch using deep generative models. Most of current drug generation models are developed upon 1D (SMILES, which is a line notation for entering and representing molecules and reactions) or 2D (molecular graph) molecular structures, which heavily rely on expensive experimental data for supervised training while ignoring the 3D interaction information between the drug and the pocket.

Recently, several drug design models have been proposed to directly generate drug molecules in the 3D space which is the realistic space of drug-target interaction. However, some of these techniques still rely on experimental data for supervised training, and others rely on performing geometric editing in the 3D molecule space guided by docking simulation. The frequent invocation of docking is very time consuming, which significantly slows down the speed of the drug design model, and docking may not always be accurate enough, especially in some complex settings.

According to aspects of the present disclosure, a method of drug design by sketching and generating (DESERT) is provided. DESERT is built on the assumption that molecular shape determines bio-activity between drug molecules and its target pocket. In other words, a drug candidate may have satisfactory bio-activity to a target pocket if their shapes are complementary.

The embodiments of the present disclosure splits a drug design process into sketching and generating stages, therefore the embodiments may not heavily rely on docking simulation, which only optionally performs docking for post-process. Furthermore, the embodiments of the present disclosure generate molecular shapes represented by three-dimension images, therefore the three-dimension interaction information between the molecules and the pockets contribute to generate molecules.

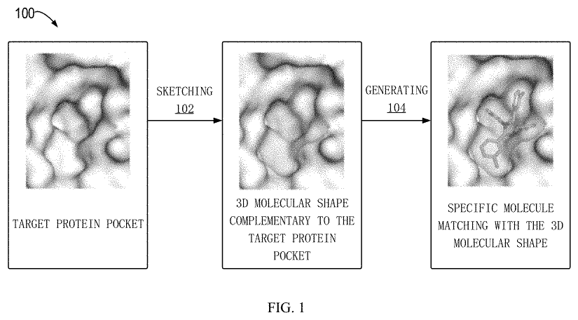

FIG. 1 illustrates a schematic process 100 that splits a drug design process into sketching and generating stages. As shown in FIG. 1, the embodiments of the present disclosure splits the whole drug design process into sketching stage 102 and generating stage 104, which employs the molecular shape as the bridge of the two stages. According to the embodiments, in the sketching stage 102, some embodiments may sample some reasonable shapes complementary to the target pocket. In the generating stage 104, the embodiments may utilize a generative pre-trained model, which converts molecular shape to concrete molecule, to fill the shape obtained in the sketching stage 102. The generative pre-trained model is trained on a database which contains sufficient pairs of molecules and corresponding molecular shapes. With such splitting, one advantage is the method may not heavily rely on docking simulation, which only optionally performs docking for post-process and thus avoids the disadvantages described above. Additionally, the method abandons the expensive experimental data. This process may not rely on experimental data, thus the embodiments of the present disclosure may work in a zero-shot fashion.

Some embodiments of the present disclosure may design drugs by first sampling appropriate shapes complementary to the target pocket and then mapping the shapes to specific molecules. Specifically, the embodiments may produce molecules in a two-stage fashion: sampling the shape of the desired drug first (sketching) and generating molecules conditioned on the resulting shape (generating). In sketching stage 102, in some embodiments, no reference protein ligands are available when the embodiments need to sample molecular shapes. The embodiments sample reasonable shapes from protein pockets based on biological observations. In some embodiments, reference protein ligands are available, such that the embodiments may reuse the shape of the reference protein ligands to design a novel one.

In generating stage 104, the embodiments of the present disclosure may generate molecules by utilizing a pre-trained generative model referred to as SHAPE2MOL. SHAPE2MOL is an encoder-decoder network mapping a shape to diverse and high-quality 3D molecules. SHAPE2MOL is trained by utilizing massive unbound molecules, thus no information about proteins is needed.

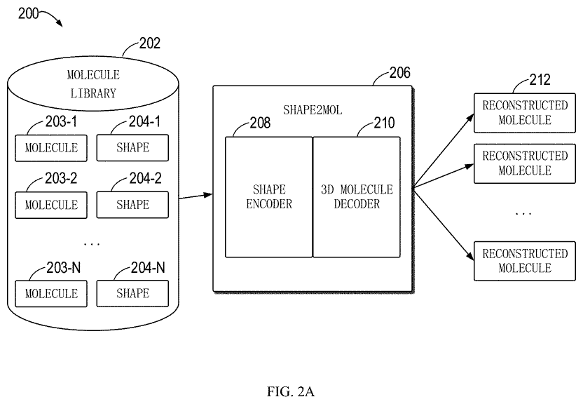

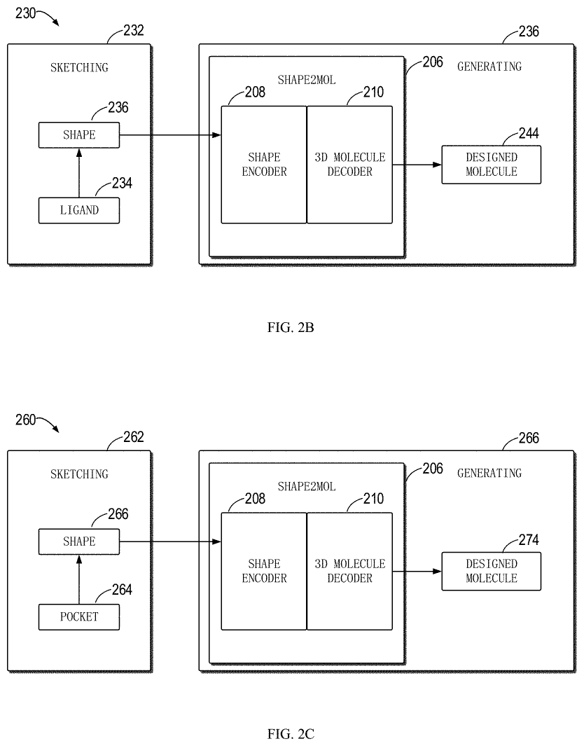

FIGS. 2A-C illustrate a training process 200 for SHAPE2MOL model 206, example drug design process 230 with SHAPE2MOL model 206 based on ligand 234, and example drug design process 260 with SHAPE2MOL model 206 based on pocket 264 in accordance with embodiments of the present disclosure. As shown in FIG. 2A, molecule library 202 is a molecule library containing plenty of molecules 203 (for example, 1000M molecules) and corresponding shapes 204. For example, shape 204-1 is corresponding to molecule 203-1, shape 204-2 is corresponding to molecule 203-2, and shape 204-N is corresponding to molecule 203-N. The molecules 203 and corresponding shapes 204 in molecule library 202 are obtained non-experimentally. The molecule library 202 may be used for training a large-scale pre-trained generative model SHAPE2MOL 206 that has a shape encoder 208 and a 3D molecule decoder 210. The shape encoder 208 receives molecular shapes 204 and encodes the molecular shapes 204 into latent information, and the 3D molecule decoder 210 then performing reconstruction to obtain reconstructed molecules 212 which contains specific molecular structure.

In some embodiments, a number of unbound molecules (for example, 100M) and their corresponding shapes may be sampled from the lead-like subset of molecule library 202 as the training data. The sampled molecule may be voxelized as a 3D image containing a sequence of 3D patches. The sequence of 3D patches may be delivered into the shape encoder 208. The shape encoder 208 then convert the sequence of 3D patches into latent information of each 3D patch, which contain the geometric information of the sampled molecule. The latent information may be served as the context of 3D molecule decoder 210 to constrain the shape of reconstructed molecules 212.

In some embodiments, in order to train the 3D molecule decoder 210, a molecule of the sampled molecules may be segmented into pieces, and then the pieces may be converted into a sequence. In some embodiments, the training process may be divided into tokenization and linearization. The embodiments first tokenize the molecule of the sampled molecules based on some principles, for example, preserving the functional groups since they are vital for determining molecule properties. Therefore, the molecule represented by graph may be converted into tree structure, so that the generative process can be easily factorized. After tokenization, the embodiments perform linearization to further convert the tree structure of the molecule into fragment sequence. Representing the molecules by fragment sequences is not only convenient for training, but also has strong power to represent any complicated tree structure. In some embodiments, the linearization may be performed by traversing the tree and adding specific symbols at beginning and ending of a branch.

In the embodiments as described above, the 3D molecule decoder 210 may be trained to predict the fragment category, rotation and translation, such that the SHAPE2MOL model 206 may learn that how exactly a fragment is placed in 3D space, and then the SHAPE2MOL model 206 may generate the next fragment connected with the current fragment. As results, a reconstructed molecule 212 may be reconstructed by connecting all fragments together.

The embodiments of the present disclosure train generative model on an existing database which contains sufficient pairs of molecules and corresponding molecular shapes, therefore the embodiments may not rely on expensive experimental data. Furthermore, the embodiments of the present disclosure utilize a pre-trained model which is aware of the shape of the pocket, therefore the performance of the design process may be improved. In addition, the embodiments of the present disclosure utilize massive unbound molecules, therefore the learned space maybe denser and the embodiments may generate diverse molecules.

As described above, in sketching stage 102, in some embodiments, reference protein ligands are available, thus the embodiments may obtain molecular shapes based on the reference protein ligands. In some embodiments, reference protein ligands are unavailable, thus the embodiments may sample molecular shapes based on protein pockets.

FIG. 2B illustrates an example drug design process 230 with SHAPE2MOL model based on ligand in accordance with embodiments of the present disclosure. In the embodiment, in sketching stage 232, a reference ligand 234 is available. A molecular shape 236 may be obtained from the reference ligand 234, for example, by obtaining a shape of the reference ligand 234 and reusing the shape of the reference ligand 234. In some embodiments, molecular shapes 236 may be obtained by searching molecule libraries (for example, molecule library 202 as shown in FIG. 2A) for molecules with similar 3D shapes but novel 2D chemical structure compared to the reference ligand 234.

In generating stage 236, the molecular shape 236 then may be delivered into the trained model SHAPE2MOL 206. In some embodiments, the molecular shape 236 may be voxelized as a 3D image containing a sequence of 3D patches. The shape encoder 208 then convert the sequence of 3D patches into latent information of each 3D patch, which contain the geometric information of the molecular shape 236. The latent information may be served as the context of 3D molecule decoder 210 to constrain the shape of designed molecules 244. In some embodiments, the 3D molecule decoder 210 may predict the fragment category, rotation and translation based on the latent information, such that the SHAPE2MOL model 206 may learn that how exactly a fragment is placed in 3D space, and then the SHAPE2MOL model 206 may generate the next fragment connected with the current fragment. As a result, the designed molecule 244 may be generated.

FIG. 2C illustrates an example drug design process 260 with SHAPE2MOL model based on pocket in accordance with embodiments of the present disclosure. In the embodiment, in sketching stage 232, a reference ligand is not provided. Therefore, a molecular shape 266 may be generated based on the pocket shape 264. In some embodiments, the molecular shape 266 may be generated based on the pocket shape 264 and a seed shape. The seed shape may be obtained by utilizing the molecular shapes in molecule libraries (for example, molecule library 202 as shown in FIG. 2A). In some embodiments, the molecular shape 266 may be generated by intersecting the seed shape with the pocket shape 264 in different ways. For example, the seed shape and the pocket shape 264 may be placed at different initial positions, the seed shape may move along with different directions or with different stop conditions.

In generating stage 266, the molecular shape 266 may be delivered into the trained model SHAPE2MOL 206. Similar with the process 230, in some embodiments, the molecular shape 266 may be voxelized as a 3D image containing a sequence of 3D patches. The shape encoder 208 then convert the sequence of 3D patches into latent information of each 3D patch, which contain the geometric information of the molecular shape 266. The latent information may be served as the context of 3D molecule decoder 210 to constrain the shape of designed molecules 274. In some embodiments, the 3D molecule decoder 210 may predict the fragment category, rotation and translation based on the latent information, such that the SHAPE2MOL model 206 may learn that how exactly a fragment is placed in 3D space, and then the SHAPE2MOL model 206 may generate the next fragment connected with the current fragment. As a result, the designed molecule 274 may be generated.

According to the embodiments of the present disclosure, the sketching stage 102 as shown in FIG. 1 is responsible for deciding what desired molecules look like. In some embodiments, a ligand for a protein pocket is available, such that a ligand-based sketching is possible. The embodiments of the present disclosure may sample reasonable shapes from protein pockets based on biological observations. In addition, the embodiments may further reuse the shape of a ligand to design a novel molecule. Since molecules with similar shapes have similar properties, it is reasonable to directly use the shape of the ligand as the shape of the desired molecule. In some embodiments, molecular shapes may be obtained by searching molecule libraries (for example, molecule library 202 as shown in FIG. 2A) for molecules with similar 3D shapes but novel 2D chemical structure compared to the reference ligand.

In some embodiments, the ligand of the pocket is unavailable, thus an approach for pocket-based sketching is provided in the present disclosure. The embodiments may obtain a shape that has a high potential to bind to a pocket based on two observations. One observation is ligands mainly lie in the area close to the pocket surface. In another word, the shape of satisfactory ligand may tightly complementary to the pocket. Another observation is pockets are usually much larger than ligands, such that directly utilizing the shape of pockets to design molecules is inappropriate. According to these two observations, the pocket-based sketching described herein samples a region with an appropriate size complementary to the surface of the pocket. Some embodiments of the present disclosure provide an approach for obtaining the desired molecule shape which is of the appropriate size and complementary to the pocket surface. The embodiments obtain a seed shape that intersects with the pocket, where the intersection has a similar size to a molecule.

The embodiments explore the protein pockets by generating multiple molecular shapes from the protein pockets, therefore, the embodiments has the potential to obtain diverse and high quality molecules that bind to protein pockets in different regions, rather than only consider one region that the ligands lie in which limits the exploration of protein pockets.

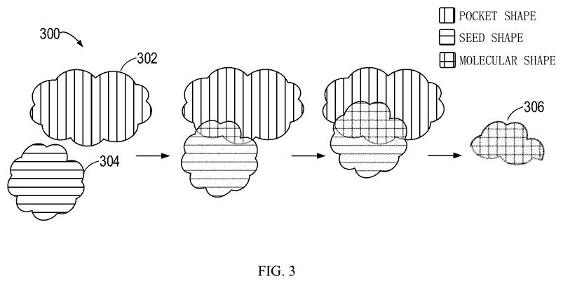

FIG. 3 illustrates an example process 300 for sampling molecular shapes from pocket in accordance with embodiments of the present disclosure. FIG. 3 shows a pocket shape 302 and a seed shape 304. Although the pocket shape 302 and the seed shape 304 are depicted as two-dimension, both of them described herein are three-dimension shape. To obtain the seed shape 304, the process 300 samples several molecules from an existing molecule library (for example, the molecule library 202 as shown in FIG. 2A) which contains sufficient pairs of molecule and corresponding shape. The process 300 then uses the overlapping of the shapes of the sampled several molecules as the seed shape 304. By using the overlapping strategy, the sketched pseudo molecular shapes 306 are more native-molecule-like and not overly dependent on one specific molecule.

After obtaining the seed shape 304, the process 300 intersects the seed shape 304 with the pocket shape 302 gradually, as shown in FIG. 3. Since the sizes of pockets are much larger than the sizes of ligands, the intersecting stops when the volume of the intersection part satisfies a threshold value. In some embodiments, average volume (for example, 300 Å³) of molecules in the molecule library may be used as the threshold value. Furthermore, the step size may be the same as a voxel resolution (for example, 0.5 Å). In addition, the seed shape 304 may be placed in a random position initially as long as the seed shape and the pocket shape do not overlap. As the result, the intersection part may be used as the molecular shape.

Such pocket-based sketching has many benefits. For example, instead of using the molecular shapes of reference ligands as input, the embodiments of the present disclosure may explore the protein pockets by sampling multiple molecular shapes from them. Therefore, the embodiments have the potential to obtain diverse and high quality molecules that bind to protein pockets in different regions, rather than only consider one region that the ligands lie in which limits the exploration of protein pockets.

Now moving forward to generating stage 104. SHAPE2MOL model is an encoder-decoder network mapping a shape to diverse and high-quality 3D molecules. SHAPE2MOL is trained by utilizing massive unbound molecules, thus no information about proteins is needed. The SHAPE2MOL model is a model related to image-to-sequence generation, where the shape is voxelized as a 3D image and the 3D molecular is converted to be a sequence. This generative approach is capable of modeling any complicated molecule structure and the linearization makes large-scale pre-training easier to implement.

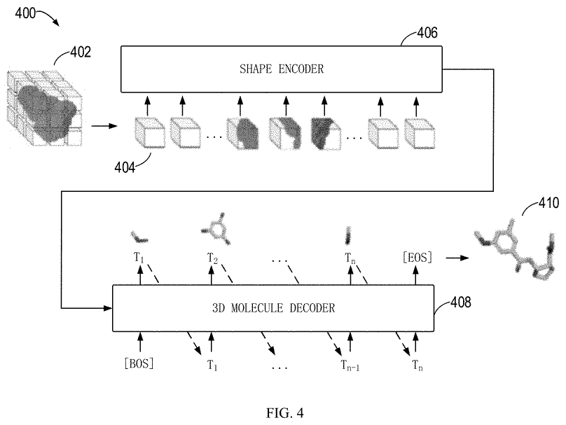

FIG. 4 illustrates architecture 400 of SHAPE2MOL model with shape encoder 406 and 3D molecule decoder 408. An encoder-decoder model is a neural network translation model that uses the mechanisms of attention, differentially weighting the significance of each part of the input data. Encoder-decoder model is designed for text-based tasks originally, for example, the encoder-decoder model can be trained to translate a sentence in a language into a sentence in another language. The encoder extracts features from an input, and the decoder uses the features to produce an output. Recently, the encoder-decoder model has been extended for 2D-image-based tasks. For example, the encoder-decoder model represents an input 2D image as a series of 2D image patches and directly predicts class labels for the input 2D image. According to the embodiments of present disclosure, in order to utilize the 3D interaction information between the molecules and pockets, the shape encoder 406 and the 3D molecule decoder 408 are extended for 3D molecular shapes.

According to the embodiments of present disclosure, a molecular shape (for example, molecular shape 266 as shown in FIG. 3) may be voxelized as a 3D molecular image 402. The voxelized 3D molecular image 402 contains a sequence of 3D image patches 404, and each 3D image patch 404 contains several voxels. Voxels in 3D image is similar with pixels in 2D image.

In some embodiments, the resolution of the voxelized 3D molecular image 402 may be set to, for example, 0.5 Å and the side length of the spanned cube may be set to, for example, 14 Å. In some embodiments, for example, the size of 3D image patches may be 4×4×4, which allows to handle a large number of voxels.

In some embodiments, in order to voxelize the molecular shape (for example, molecular shape 266 as shown in FIG. 2C), the embodiments use [symbol] to denote the set of all atoms. A molecule m may be constructed as a collection of atoms and their corresponding coordinates. The molecule m may be represented as the following Equation 1:

m={(α,c)|α∈ [symbol] ,c∈ [symbol] ³} (Equation 1)

Given a molecule m, the shape encoder 406 transforms its shape into a 3D image with a voxelization function vₘ: [symbol] →{0,1}. The 3D image may be represented as the following Equation 2:

$${v}_{m} ( x , y , z ) = \{ \begin{matrix}1 & ∃ ( a , c ) ∈ m , {‖ ( x , y , z ) - c ‖}_{2} \leq r ( a ) + ϵ \\ 0 & otherwise\end{matrix} \qquad ( Equation 2 )$$

Where r denotes the Van der Waals radii, ∈ is a perturbed noise which helps prevent overfitting.

In FIG. 4, the output of the shape encoder 406 is latent information of the 3D image patches 404 and the latent information includes the continuous representation of each 3D image patch, which contains the geometric information of input molecular shape 402. The output may serve as the context of the decoder 408 to constrain the shape of generated 3D molecules 410. In the embodiments, the 3D molecule decoder 408 receives the latent information generated by the shape encoder 406, and then generates fragments of molecule based on the latent information. According to the embodiments, the input of the 3D molecule decoder 408 at the time step t is the fragment category, rotation quaternion, and translation vector from the output of the previous step t-1. Therefore, the 3D molecule decoder 408 may learn how exactly a fragment is placed in 3D space, so that the 3D molecule decoder 408 may generate the next fragment connected with it. The output of the shape encoder 406 is fed into the 3D molecule decoder 408 as the geometric context, through a cross-attention module. After generating the fragments, they may be connected to reconstruct a 3D molecule 410.

In some embodiments, in order to stabilize the training process, the translation vector and rotation quaternion may be discretized. According to the embodiments, the translation vector and rotation quaternion are mapped into two discrete spaces. The discrete space for the translation vector is grids in 3D space. This continuous vector is represented with the discrete index of the grid. The i-th translation bin is represented as the coordinate of its center tᵢ^{bin}∈ [symbol] ³. The discretization of any continuous translation operator t∈ [symbol] ³ can be computed by arg min∥tᵢ^{bin}, t∥₂. Because the rotation axis and rotation angle can determine the rotation quaternion, the discrete space of the quaternion contains two parts. The rotation axes (x, y, z) are enumerated in 3D space, then for each axis, every θ degrees of rotation angle are considered. The i-th rotation bin is represented as a quaternion qᵢ^{bin}=(xᵢ, yᵢ, zᵢ, θᵢ)∈ [symbol] ⁴. The discretization of any continuous rotation operator q∈ [symbol] ⁴ can be computed by ∥qᵢ^{bin}, q∥₂. Another reason for the discretization is to avoid the discontinuity of quaternions when optimizing them. The embodiments of present disclosure may convert a regression problem into a classification one with discretization, thus avoiding the discontinuity of quaternions issue.

In some embodiments, a greedy algorithm may be used for connecting fragments. The algorithm converts the separated fragments into a complete 3D molecule. According to the embodiments, the fragments may be placed in 3D space according to the predictions of SHAPE2MOL. Then, for each time, two closest breakpoints are chose greedily from different fragments and the fragments may be connected through the breakpoints. The fragments get larger and larger by repeating the process. The largest fragment may be returned as the final molecule when there are not enough breakpoints to connect. In some embodiments, for potential residual breakpoints, carbon atoms may be attached to these breakpoints for molecular validity.

In some embodiments, some post processing operations may be performed to further improve the diversity and quality. For example, the duplicate molecules may be removed to leverage the docking simulation to drop molecules that do not pass the affinity threshold.

Generating 3D molecules utilizing the embodiments of the present disclosure with SHAPE2MOL has many benefits. For example, the molecules generated by the embodiments have high quality, since SHAPE2MOL is aware of the shape of pocket, such that the generated molecules complement the pocket better than the 3D molecular in existing molecular library. Furthermore, both of the molecular shape and the pocket shape are represented by three-dimension, such that SHAPE2MOL may take the 3D interaction information between the molecules and the pockets into consider, while the tradition drug design approaches and other generative models may ignore these 3D interaction information.

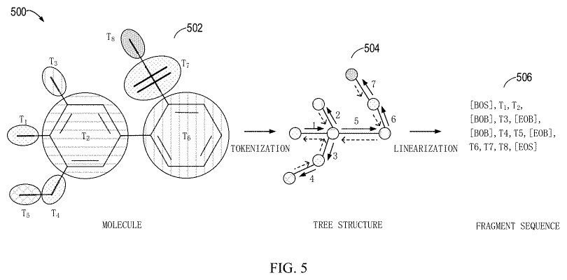

In the embodiments of the present disclosure, 3D molecule decoder 408 as shown in FIG. 4 handles a 3D molecule as a sequence of tuples. The sequence object eases the implementation of a pre-trained model. Therefore, in order to train the 3D molecule decoder 408, fragment sequences representing molecules may be generated as training data. FIG. 5 illustrates an example process 500 for converting a molecule 502 into a fragment sequence 506 in accordance with embodiments of the present disclosure. In some embodiments, in order to obtain the object, the 3D molecule decoder 408 segments a molecule 502 into fragments, then convert it to a fragment sequence 506.

As shown in FIG. 5, the 3D molecule 502 is tokenized into a tree structure 504. In other word, the molecule 502 may be segmented into fragments, such that the generative process can be easily factorized. In some embodiments, the segmenting is based on the functional group of the molecule 502, the size of remaining fragments and the graph structure of the remaining fragments. In some embodiments, the molecule 502 may be segmented by one or more principles.

In some embodiments, the molecule 502 may be segmented while preserving the functional groups since they are vital for determining molecule properties. Otherwise, the molecular structures that are vital for determining molecule properties may be ruined without distinguishing a chemical bond belonging to a functional group. In some embodiments, the molecule 502 may be segmented while avoiding too large size of the vocabulary to ease the pre-training process. In some embodiments, the molecule 502 may be segmented while no circles exist in the segmented molecules since a tree structure is simpler to handle than a graph. In some embodiments, the molecule 502 may be segmented while cutting all single bonds attached to a ring since over 60% of the out-of-vocabulary problem, an essential factor affecting the pre-trained model quality, is caused by the combinations between rings and other structures through single bonds.

As shown in FIG. 5, after tokenization, the tree structure 504 of the molecule is linearized into fragment sequence 506. A linearized sequence which is the output of the network may be utilized to represent the target molecule graph. One advantage of linearized sequence is convenient for training, and another advantage is linearized sequence has a strong power to represent complicated tree structure. In some embodiments, the fragment with degree 1 may be selected as the root of the tree (for example, T1, T3, T5 and T8 in FIG. 5). Then the tree may be traversed in the depth first search style. In some embodiments, a symbol [BOB] may be added at the beginning of a branch when entering the branch and a symbol [EOB] may be added at the ending of a branch when leaving the branch.

In some embodiments, a tuple (C, P, R) may be used to represent a fragment F, where C=1_{F} is an indicator function denoting its index in the vocabulary, P∈ [symbol] ³ is the translation vector, R∈ [symbol] ¹ is the rotation quaternion. In order to stabilize the training process, the continuous variables P and R are further discretized into P^{C} and R^{C}, respectively. For example, the translation vector P may be converted into a binary vector P^{C}, which satisfies the Equation 3:

$${P}^{c} [ i ] = \{ \begin{matrix}1 & ⌊ \frac{L}{b} i ⌋ \leq P < ⌈ \frac{L}{b} i ⌉ \\ 0 & otherwise\end{matrix} \qquad ( Equation 3 )$$

Where L is the max translation length, b is the bin size.

In some embodiments, the training data may include molecules, corresponding molecular shapes and corresponding fragment sequences. The molecules and corresponding molecular shapes may be obtained from the molecule library 202 in FIG. 2A. The corresponding fragment sequences may be generated by performing the process of converting molecule into sequence described in FIG. 5. Furthermore, the model may be trained using the output probabilities of the model (Ĉᵢ, \hat{P}ᵢ^{c}, \hat{R}ᵢ^{c}), which denote the probability of a fragment, a discretized translation vector, and a discretized rotation vector, respectively. Corresponding cross-entropy loss may be calculated and their sum may be used as the final loss function. The loss may be represented as the following Equation 4:

$$ℒ = - {\sum }_{i = 1}^{n} \{ {C}_{i} log {\hat{C}}_{i} + {P}_{i}^{c} log {\hat{P}}_{i}^{c} + {R}_{i}^{c} log {\hat{R}}_{i}^{c} \} \qquad ( Equation 4 )$$

Where n is the length of the fragment sequence.

The embodiments of the present disclosure may utilize massive unbound molecules, which leads to the learned space being denser. Combing with an appropriate sampling method, the embodiments may generate diverse molecules. Comparing to the embodiments of the present disclosure, traditional supervised methods easily collapse to the main molecule pattern in their database, since the labeled data is inadequate.

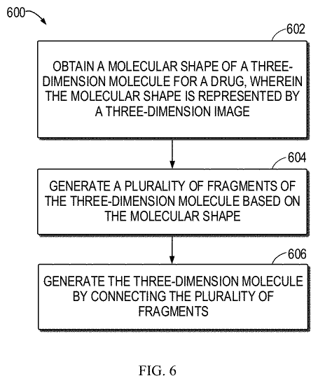

FIG. 6 illustrates a flowchart of a method 600 for designing drug that splits a drug design process into sketching and generating stages.

At 602, the method 600 obtains a molecular shape of a three-dimension molecule for a drug, wherein the molecular shape is represented by a three-dimension image. For example, in the embodiments which reference ligand is unavailable, as shown in FIG. 3, the method 600 may use a three-dimension seed shape 304 to intersect with a three-dimension pocket shape 302 gradually, and may obtain a three-dimension molecular shape 306 which is the intersection part as result. In the embodiments, the seed shapes 304 may be generated from existing molecule libraries.

At 604, the method 600 generates a plurality of fragments of the three-dimension molecule based on the molecular shape. For example, as shown in FIG. 4, the molecular shape 402 may be voxelized into voxels 404. The shape encoder may convert the voxels 404 into latent information containing the geometric information of the voxels 404. The 3D molecule decoder may translate the latent information and generate a plurality of fragments, for example, T₁, T₂ and Tₙ.

At 606, the method 600 generates the three-dimension molecule by connecting the plurality of fragments. For example, as shown in FIG. 4, the 3D molecule 410 may be generated by connecting the plurality of fragments (for example, T₁, T₂ and Tₙ) together.

The method 600 according to embodiments of the present disclosure may be used to design high quality drugs for specific protein pockets efficiently. Finding appropriate candidate molecules for specific protein pockets is an important step in drug design, therefore finding rational drug molecules efficiently may speed up the process of drug development and reduce the cycle of drug development. Furthermore, since method 600 utilizes large-scale non-experimental data, method 600 may not rely on expensive experimental data and docking simulation which is time consuming. Additionally, method 600 utilizes the three-dimension interaction information between the molecules and the pockets to generate drug molecules, thus the quality of generated drug molecules is also improved.

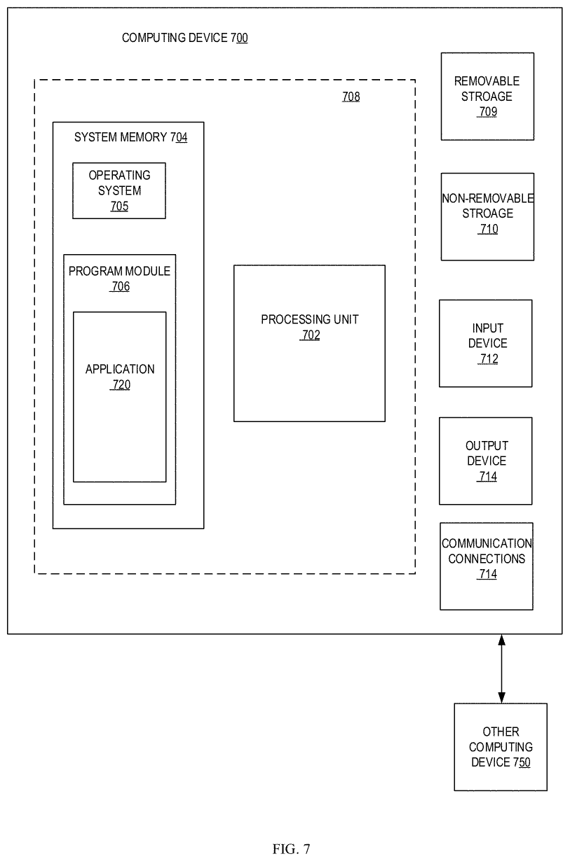

FIG. 7 is a block diagram illustrating physical components (e.g., hardware) of an electronic device 700 with which aspects of the disclosure may be practiced. For example, the electronic device 700 may implements the processes as depicted in FIGS. 1-6. In a basic configuration, the processing device 700 may include at least one processing unit 702 and a system memory 704. Depending on the configuration and type of computing device, the system memory 704 may comprise, but is not limited to, volatile storage (e.g., random access memory), non-volatile storage (e.g., read-only memory), flash memory, or any combination of such memories.

The system memory 704 may include an operating system 705 and one or more program modules 706 suitable for performing the various aspects disclosed herein such. The operating system 705, for example, may be suitable for controlling the operation of the processing device 700. Furthermore, aspects of the disclosure may be practiced in conjunction with other operating systems, or any other application program and is not limited to any particular application or system. This basic configuration is illustrated in FIG. 7 by those components within a dashed line 708. The processing device 700 may have additional features or functionality. For example, the processing device 700 may also include additional data storage devices (removable and/or non-removable) such as, for example, magnetic disks, optical disks, or tape. Such additional storage is illustrated in FIG. 7 by a removable storage device 709 and a non-removable storage device 710.

As stated above, several program modules and data files may be stored in the system memory 704. While executing on the at least one processing unit 702, an application 720 or program modules 706 may perform processes including, but not limited to, one or more aspects, as described herein. The application 720 may include an application interface 721 which may be the same as or similar to the application interface 721 as previously described in more detail with regard to FIGS. 1-6. Other program modules that may be used in accordance with aspects of the present disclosure may include electronic mail and contacts applications, word processing applications, spreadsheet applications, database applications, slide presentation applications, drawing or computer-aided application programs, etc., and/or one or more components supported by the systems described herein.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 7 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the processing device 500 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general-purpose computer or in any other circuits or systems.

The processing device 700 may also have one or more input device(s) 712 such as a keyboard, a mouse, a pen, a sound or voice input device, a touch or swipe input device, etc. The output device(s) 714 such as a display, speakers, a printer, etc. may also be included. The aforementioned devices are examples and others may be used. The processing device 500 may include one or more communication connections allowing communications with other computing or processing devices 750. Examples of suitable communication connections include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 904, the removable storage device 709, and the non-removable storage device 710 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the processing device 700. Any such computer storage media may be part of the processing device 700. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

In addition, the aspects and functionalities described herein may operate over distributed systems (e.g., cloud-based computing systems), where application functionality, memory, data storage and retrieval and various processing functions may be operated remotely from each other over a distributed computing network, such as the Internet or an intranet. User interfaces and information of various types may be displayed via on-board computing device displays or via remote display units associated with one or more computing devices. For example, user interfaces and information of various types may be displayed and interacted with. Interaction with the multitude of computing systems with which embodiments of the invention may be practiced include, keystroke entry, touch screen entry, voice or other audio entry, gesture entry where an associated computing device is equipped with detection (e.g., camera) functionality for capturing and interpreting user gestures for controlling the functionality of the computing device, and the like.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Any of the steps, functions, and operations discussed herein can be performed continuously and automatically.

The exemplary systems and methods of this disclosure have been described in relation to computing devices. However, to avoid unnecessarily obscuring the present disclosure, the preceding description omits several known structures and devices. This omission is not to be construed as a limitation. Specific details are set forth to provide an understanding of the present disclosure. It should, however, be appreciated that the present disclosure may be practiced in a variety of ways beyond the specific detail set forth herein.

Furthermore, while the exemplary aspects illustrated herein show the various components of the system collocated, certain components of the system can be located remotely, at distant portions of a distributed network, such as a LAN and/or the Internet, or within a dedicated system. Thus, it should be appreciated, that the components of the system can be combined into one or more devices, such as a server, communication device, or collocated on a particular node of a distributed network, such as an analog and/or digital telecommunications network, a packet-switched network, or a circuit-switched network. It will be appreciated from the preceding description, and for reasons of computational efficiency, that the components of the system can be arranged at any location within a distributed network of components without affecting the operation of the system.

Furthermore, it should be appreciated that the various links connecting the elements can be wired or wireless links, or any combination thereof, or any other known or later developed element(s) that is capable of supplying and/or communicating data to and from the connected elements. These wired or wireless links can also be secure links and may be capable of communicating encrypted information. Transmission media used as links, for example, can be any suitable carrier for electrical signals, including coaxial cables, copper wire, and fiber optics, and may take the form of acoustic or light waves, such as those generated during radio-wave and infra-red data communications.

While the flowcharts have been discussed and illustrated in relation to a particular sequence of events, it should be appreciated that changes, additions, and omissions to this sequence can occur without materially affecting the operation of the disclosed configurations and aspects.

Several variations and modifications of the disclosure can be used. It would be possible to provide for some features of the disclosure without providing others.

In yet another configurations, the systems and methods of this disclosure can be implemented in conjunction with a special purpose computer, a programmed microprocessor or microcontroller and peripheral integrated circuit element(s), an ASIC or other integrated circuit, a digital signal processor, a hard-wired electronic or logic circuit such as discrete element circuit, a programmable logic device or gate array such as PLD, PLA, FPGA, PAL, special purpose computer, any comparable means, or the like. In general, any device(s) or means capable of implementing the methodology illustrated herein can be used to implement the various aspects of this disclosure. Exemplary hardware that can be used for the present disclosure includes computers, handheld devices, telephones (e.g., cellular, Internet enabled, digital, analog, hybrids, and others), and other hardware known in the art. Some of these devices include processors (e.g., a single or multiple microprocessors), memory, nonvolatile storage, input devices, and output devices. Furthermore, alternative software implementations including, but not limited to, distributed processing or component/object distributed processing, parallel processing, or virtual machine processing can also be constructed to implement the methods described herein.

In yet another configuration, the disclosed methods may be readily implemented in conjunction with software using object or object-oriented software development environments that provide portable source code that can be used on a variety of computer or workstation platforms. Alternatively, the disclosed system may be implemented partially or fully in hardware using standard logic circuits or VLSI design. Whether software or hardware is used to implement the systems in accordance with this disclosure is dependent on the speed and/or efficiency requirements of the system, the particular function, and the particular software or hardware systems or microprocessor or microcomputer systems being utilized.

In yet another configuration, the disclosed methods may be partially implemented in software that can be stored on a storage medium, executed on programmed general-purpose computer with the cooperation of a controller and memory, a special purpose computer, a microprocessor, or the like. In these instances, the systems and methods of this disclosure can be implemented as a program embedded on a personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

The disclosure is not limited to standards and protocols if described. Other similar standards and protocols not mentioned herein are in existence and are included in the present disclosure. Moreover, the standards and protocols mentioned herein, and other similar standards and protocols not mentioned herein are periodically superseded by faster or more effective equivalents having essentially the same functions. Such replacement standards and protocols having the same functions are considered equivalents included in the present disclosure.

The present disclosure, in various configurations and aspects, includes components, methods, processes, systems and/or apparatus substantially as depicted and described herein, including various combinations, sub-combinations, and subsets thereof. Those of skill in the art will understand how to make and use the systems and methods disclosed herein after understanding the present disclosure. The present disclosure, in various configurations and aspects, includes providing devices and processes in the absence of items not depicted and/or described herein or in various configurations or aspects hereof, including in the absence of such items as may have been used in previous devices or processes, e.g., for improving performance, achieving ease, and/or reducing cost of implementation.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

1. A method, comprising:
obtaining a molecular shape of a three-dimension molecule for a drug, wherein the molecular shape is represented by a three-dimension image;
generating a plurality of fragments of the three-dimension molecule based on the molecular shape; and
generating the three-dimension molecule by connecting the plurality of fragments.

2. The method of claim 1, wherein obtaining a molecular shape of a three-dimension molecule for a drug comprises:
determining a ligand shape of a given ligand; and
obtaining the molecular shape based on the ligand shape.

3. The method of claim 1, wherein obtaining a molecular shape of a three-dimension molecule for a drug comprises:
obtaining a seed shape and a pocket shape of a protein target pocket; and
determining the molecular shape based on the seed shape and the pocket shape.

4. The method of claim 3, wherein obtaining the seed shape comprises:
sampling a plurality of molecules from a molecule library; and
generating the seed shape by overlapping shapes of the sampled plurality of molecules.

5. The method of claim 4, wherein determining the molecular shape based on the seed shape and the pocket shape comprises:
intersecting the seed shape with the pocket shape until a volume of overlapped part of the seed shape and the pocket shape satisfies a threshold value; and
obtaining the molecular shape based on the intersection of the seed shape and the pocket shape.

6. The method of claim 5, wherein the threshold value is average volume of molecules in the molecule library.

7. The method of claim 1, wherein generating a plurality of fragments of the three-dimension molecule based on the molecular shape comprises:
voxelizing, by a shape encoder, the molecular shape to generate a plurality of voxels;
generating, by the shape encoder, latent information of the plurality of voxels; and
generating, by a three-dimension molecule decoder and based on the latent information, the plurality of fragments.

8. The method of claim 7, further comprising:
obtaining a first molecule from a molecule library;
generating a tree structure of the first molecule by tokenizing the first molecule; and
linearizing the tree structure into a fragment sequence.

9. The method of claim 8, wherein tokenizing the first molecule comprises:
segmenting the first molecule into one or more fragments based on at least one of a functional group of the first molecule, a size of a fragment, or a graph structure of a fragment.

10. The method of claim 8, wherein linearizing the tree structure into a fragment sequence comprises:
traversing the tree structure in depth first search;
adding a first symbol into the linearized fragment sequence when entering a branch of the tree structure; and
adding a second symbol into the linearized fragment sequence when leaving the branch of the tree structure, wherein the second symbol is different from the first symbol.

11. The method of claim 8, further comprising:
generating a first molecular shape of the first molecule; and
training the shape encoder and the three-dimension molecule decoder using the first molecular shape, the first molecule and the fragment sequence.

12. An electronic device, comprising:
a memory and a processor;
wherein the memory is used to store one or more computer instructions which, when executed by the processor, cause the processor to perform the actions comprising:
obtaining a molecular shape of a three-dimension molecule for a drug, wherein the molecular shape is represented by a three-dimension image;
generating a plurality of fragments of the three-dimension molecule based on the molecular shape; and
generating the three-dimension molecule by connecting the plurality of fragments.

13. The electronic device of claim 12, wherein obtaining a molecular shape of a three-dimension molecule for a drug comprises:
determining a ligand shape of a given ligand; and
obtaining the molecular shape based on the ligand shape.

14. The electronic device of claim 12, wherein obtaining a molecular shape of a three-dimension molecule for a drug comprises:
obtaining a seed shape and a pocket shape of a protein target pocket; and
determining the molecular shape based on the seed shape and the pocket shape.

15. The electronic device of claim 14, wherein obtaining the seed shape comprises:
sampling a plurality of molecules from a molecule library; and
generating the seed shape by overlapping shapes of the sampled plurality of molecules.

16. The electronic device of claim 15, wherein determining the molecular shape based on the seed shape and the pocket shape comprises:
intersecting the seed shape with the pocket shape until a volume of overlapped part of the seed shape and the pocket shape satisfies a threshold value; and
obtaining the molecular shape based on the intersection of the seed shape and the pocket shape.

17. The electronic device of claim 15, wherein the threshold value is average volume of molecules in the molecule library.

18. The electronic device of claim 12, wherein generating a plurality of fragments of the three-dimension molecule based on the molecular shape comprises:
voxelizing, by a shape encoder, the molecular shape to generate a plurality of voxels;
generating, by the shape encoder, latent information of the plurality of voxels; and
generating, by a three-dimension molecule decoder and based on the latent information, the plurality of fragments.

19. The electronic device of claim 18, further comprising:
obtaining a first molecule from a molecule library;
generating a tree structure of the first molecule by tokenizing the first molecule; and
linearizing the tree structure into a fragment sequence.

20. A non-transitory computer-readable medium comprising instructions stored thereon which, when executed by an apparatus, cause the apparatus to perform acts comprising:
obtaining a molecular shape of a three-dimension molecule for a drug, wherein the molecular shape is represented by a three-dimension image;
generating a plurality of fragments of the three-dimension molecule based on the molecular shape; and
generating the three-dimension molecule by connecting the plurality of fragments.
